(19) United States
(12) Patent Application Publication
(10) Pub. No.: US 2025/0360245 A1
(43) Pub. Date: Nov. 27, 2025

(54) CLEANABLE AND SMART AEROGEL WOUND DRESSINGS

(71) Applicants: Aparna Arun Agrawal, North Olmsted, OH (US); Isaiah Kaiser, Akron, OH (US); Sadhan Jana, Fairlawn, OH (US)

(72) Inventors: Aparna Arun Agrawal, North Olmsted, OH (US); Isaiah Kaiser, Akron, OH (US); Sadhan Jana, Fairlawn, OH (US)

(21) Appl. No.: 18/873,457

(22) PCT Filed: Apr. 10, 2023
(86) PCT No.: PCT/US2023/018031

§ 371 Date: Dec. 10, 2024

Related U.S. Application Data
(60) Provisional application No. 63/350,963, filed on Jun. 10, 2022.

(51) Int. Cl. A61L 26/00

(52) U.S. Cl. CPC A61L 26/008; A61L 26/0023; A61L 26/0061; A61L 26/0085

(57) ABSTRACT

An aerogel wound dressing including one or more aerogel layers, including a sensing component for monitoring a wound environment, and methods for treating a wound using an aerogel wound dressing. The wound dressing includes a base layer including which may include a sensing component, and optional removable layers which may be removed upon saturation of a wound exudate when used to treat a wound. The methods for treating a wound include applying an aerogel wound dressing to a wound, monitoring for infection, replacing the wound dressing upon infection, and replacing the removable layers upon saturation of wound exudate.

### CROSS-REFERENCE TO RELATED APPLICATIONS

This application claims the benefit of U.S. Provisional Patent Application Ser. Number U.S. 63/350,963 filed Jun. 10, 2022, which is incorporated herein by reference in its entirety.

### STATEMENT REGARDING FEDERALLY SPONSORED RESEARCH OR DEVELOPMENT

This invention was made with government support under CMMI-1826030 awarded by National Science Foundation. The government has certain rights in the invention.

### FIELD OF THE INVENTION

The present invention relates to the field of medical devices, specifically to wound dressings for the treatment of wounds, including chronic wounds. In particular embodiments, the present invention relates to an aerogel-based wound dressing that facilitates the healing process of wounds, including chronic wounds. In further embodiments, the invention relates to methods of treating wounds using an-aerogel based wound dressing

### BACKGROUND OF THE INVENTION

Wound healing is a complex process. Some wounds such as chronic wounds do not heal from several weeks up to years, introducing further complexity to the healing process. The objective of wound care is to accelerate healing and prevent a wound from becoming chronic. The four stages of wound healing include hemostasis, inflammation, proliferation, and remodeling. In chronic wounds, the healing process remains in the inflammation phase, where localized swelling, redness, heat, and pain are experienced by the patients. In this stage, it is common that the wound exudes a liquid composed of water, salt, proteins, fluid from injured blood vessels, debris from damaged tissues, growth factors, etc. This liquid is known as the wound exudate. Proper exudate management and removal are crucial for such wounds to heal and progress into the later stages of wound healing. Further, it is important to prevent the wound from getting infected due to pathogens from the environment. Any such infection could slow healing or cause further damage to the wound. When a wound gets infected with pathogens, it forms a ‘biofilm’ which is a membranous tissue formed by microbes that get attached to the wound surface and produce destructive enzymes and toxins that promote the inflammation phase in the wound.

In the current scenario, poor exudate absorbance of the dressings and the need for monitoring the wound for infection are some of the reasons why wound dressings are often changed daily. When a dressing is changed, it disturbs the wound bed and exposes the wound to the environment, thereby increasing the risk of biofilm formation and delayed healing. For this reason, a novel wound dressing that can absorb a large amount of exudate and can be left on the wound for a longer period is lucrative. High absorbance of exudate from the wound and restriction of bacteria/viruses from the environment are two critical characteristics that a wound dressing, especially a chronic wound dressing, should possess. Thus, there is a need for a cleanable and smart dressing with all the properties mentioned above.

### SUMMARY OF THE INVENTION

An embodiment of the present invention provides an aerogel wound dressing including an aerogel layer, wherein the aerogel layer includes a sensing component.

Another embodiment of the present invention provides an aerogel wound dressing as in any embodiment above, wherein the sensing component indicates a change in pH of a wound exudate absorbed by the aerogel layer.

Another embodiment of the present invention provides an aerogel wound dressing as in any embodiment above, wherein the sensing component changes color because of the change in pH of the wound exudate absorbed by the aerogel layer.

Another embodiment of the present invention provides an aerogel wound dressing as in any embodiment above, wherein the sensing component comprises curcumin.

Another embodiment of the present invention provides an aerogel wound dressing as in any embodiment above, wherein the sensing component is added to the first aerogel layer during formation of the first aerogel layer.

Another embodiment of the present invention provides an aerogel wound dressing as in any embodiment above, wherein the sensing component comprises a probe connected to an external sensing device.

Another embodiment of the present invention provides an aerogel wound dressing as in any embodiment above, wherein the first aerogel layer absorbs 10 times or greater exudate by weight per weight of first aerogel layer.

Another embodiment of the present invention provides an aerogel wound dressing as in any embodiment above, further comprising a second aerogel layer in contact with the first aerogel layer, wherein the second aerogel layer is removable from the aerogel wound dressing.

Another embodiment of the present invention provides an aerogel wound dressing as in any embodiment above, wherein the second aerogel layer absorbs fluids absorbed by the first aerogel layer.

Another embodiment of the present invention provides an aerogel wound dressing as in any embodiment above, wherein the first aerogel layer includes a first plurality of interconnected pores, and wherein the second aerogel layer includes a second plurality of interconnected pores.

Another embodiment of the present invention provides an aerogel wound dressing as in any embodiment above, wherein an average pore size of the first plurality of interconnected pores is larger than an average pore size of the second plurality of interconnected pores.

Another embodiment of the present invention provides an aerogel wound dressing as in any embodiment above, wherein the first aerogel layer includes a sensing component.

Another embodiment of the present invention provides an aerogel wound dressing as in any embodiment above, wherein the aerogel wound dressing is arranged such that the second aerogel layer is user replaceable upon saturation and removal of the second aerogel layer.

Another embodiment of the present invention provides an aerogel wound dressing as in any embodiment above, wherein the aerogel wound dressing is arranged such that the first aerogel layer is arranged to remain in place upon removal of the second aerogel layer.

Another embodiment of the present invention provides an aerogel wound dressing as in any embodiment above, wherein the first aerogel layer includes a sensing component.

Another embodiment of the present invention provides an aerogel wound dressing as in any embodiment above, wherein pathogenic agents cannot pass through the first aerogel layer.

Another embodiment of the present invention provides an aerogel wound dressing as in any embodiment above, wherein the first aerogel layer is formed from at least one of cellulose, cellulose nanocrystals, chitosan, Kevlar, lignin, or any combination thereof, and wherein the second aerogel layer is formed from at least one of cellulose, cellulose nanocrystals, chitosan, Kevlar, lignin, or any combination thereof.

Another embodiment of the present invention provides an aerogel wound dressing as in any embodiment above, wherein the sensing component is sensitive to a pH of a wound exudate absorbed by the aerogel wound dressing.

Another embodiment of the present invention provides an aerogel wound dressing as in any embodiment above, wherein the sensing component indicates a change in pH of the wound exudate in response to the presence of a pathogen or pathogenic byproducts in the wound exudate.

Another embodiment of the present invention provides an aerogel wound dressing as in any embodiment above, wherein the sensing component changes color because of the change in pH of the wound exudate.

Another embodiment of the present invention provides an aerogel wound dressing as in any embodiment above, wherein the sensing component exhibits keto-enol tautomerism.

Another embodiment of the present invention provides an aerogel wound dressing as in any embodiment above, wherein the sensing component comprises curcumin.

Another embodiment of the present invention provides an aerogel wound dressing as in any embodiment above, wherein the sensing component is added to the aerogel layer during formation of the aerogel layer.

Another embodiment of the present invention provides an aerogel wound dressing as in any embodiment above, wherein the sensing component comprises a probe connected to an external sensing device.

Another embodiment of the present invention provides an aerogel wound dressing as in any embodiment above, wherein the aerogel layer comprises a biopolymer.

Another embodiment of the present invention provides an aerogel wound dressing as in any embodiment above, wherein the biopolymer comprises at least one of cellulose, cellulose nanocrystals, chitosan, Kevlar, lignin, or any combination thereof.

Another embodiment of the present invention provides an aerogel wound dressing as in any embodiment above, wherein the aerogel layer comprises two or more biopolymers.

Another embodiment of the present invention provides an aerogel wound dressing as in any embodiment above, wherein the aerogel layer comprises a biopolymer gel grown inside another biopolymer gel.

Another embodiment of the present invention provides an aerogel wound dressing as in any embodiment above, wherein the aerogel layer comprises a biopolymer.

Another embodiment of the present invention provides an aerogel wound dressing as in any embodiment above, wherein the aerogel layer has a filtration efficiency of 98.9% or greater.

Another embodiment of the present invention provides an aerogel wound dressing as in any embodiment above, wherein pathogenic agents cannot pass through the aerogel layer.

Another embodiment of the present invention provides an aerogel wound dressing as in any embodiment above, wherein the aerogel layer absorbs 10 times or greater exudate by weight per weight of aerogel layer.

Another embodiment of the present invention provides an aerogel wound dressing as in any embodiment above, wherein the aerogel layer is characterized by a microstructure.

Another embodiment of the present invention provides an aerogel wound dressing as in any embodiment above, wherein the microstructure includes a plurality of interconnected pores.

Another embodiment of the present invention provides an aerogel wound dressing as in any embodiment above, wherein pores in the plurality of interconnected pores have a hole size of from about 2 nm to about 50 nm.

Another embodiment of the present invention provides an aerogel wound dressing as in any embodiment above, wherein the microstructure has a polymer domain thickness of from about 10 nm to about 50 nm.

An embodiment of the present invention provides an aerogel wound dressing including a first aerogel layer and a second aerogel layer in contact with the first aerogel layer, wherein the second aerogel layer is removable from the aerogel wound dressing.

Another embodiment of the present invention provides an aerogel wound dressing as in any embodiment above, wherein the first aerogel layer is characterized by a first microstructure.

Another embodiment of the present invention provides an aerogel wound dressing as in any embodiment above, wherein the first microstructure includes a plurality of interconnected macropores.

Another embodiment of the present invention provides an aerogel wound dressing as in any embodiment above, wherein the macropores in the plurality of interconnected macropores have a hole size of from about 50 nm to about 200 nm.

Another embodiment of the present invention provides an aerogel wound dressing as in any embodiment above, wherein the second aerogel layer is characterized by a second microstructure.

Another embodiment of the present invention provides an aerogel wound dressing as in any embodiment above, wherein the second microstructure includes a plurality of interconnected micropores.

Another embodiment of the present invention provides an aerogel wound dressing as in any embodiment above, wherein the micropores in the plurality of interconnected micropores have a hole size of from about 2 nm to about 50 nm.

Another embodiment of the present invention provides an aerogel wound dressing as in any embodiment above, wherein the first aerogel layer comprises a first biopolymer.

Another embodiment of the present invention provides an aerogel wound dressing as in any embodiment above, wherein the second aerogel layer comprises a second biopolymer.

Another embodiment of the present invention provides an aerogel wound dressing as in any embodiment above, wherein the first aerogel layer and the second aerogel layer comprise a biopolymer.

Another embodiment of the present invention provides an aerogel wound dressing as in any embodiment above, wherein the first aerogel layer comprises a first plurality of pores and the second aerogel layer comprises a second plurality of pores, wherein an average size of the first plurality of pores is the same as an average size of the second plurality of pores.

Another embodiment of the present invention provides an aerogel wound dressing as in any embodiment above, wherein the first aerogel layer comprises a first plurality of pores and the second aerogel layer comprises a second plurality of pores, wherein an average size of the first plurality of pores is larger than an average size of the second plurality of pores.

Another embodiment of the present invention provides an aerogel wound dressing as in any embodiment above, wherein the first aerogel layer includes a sensing component.

Another embodiment of the present invention provides an aerogel wound dressing as in any embodiment above, wherein the first aerogel layer has a filtration efficiency of 98.9% or greater.

Another embodiment of the present invention provides an aerogel wound dressing as in any embodiment above, wherein pathogenic agents cannot pass through the first aerogel layer.

An embodiment of the present invention provides a method of treating a wound, the method including applying an aerogel wound dressing to a wound.

Another embodiment of the present invention provides a method of treating a wound as in any embodiment above, further including the steps of monitoring the sensing component for the presence of a pathogen, removing the aerogel wound dressing from the wound when the sensing component indicates the presence of a pathogen, and applying a treatment to the aerogel wound dressing when the sensing component indicates the presence of a pathogen.

Another embodiment of the present invention provides a method of treating a wound as in any embodiment above, wherein the aerogel wound dressing is a multilayered aerogel wound dressing comprising a first aerogel layer, including the sensing component, and a second aerogel layer in contact with the first aerogel layer, wherein the second aerogel layer is removable from the aerogel wound dressing; and the method further comprising removing the second aerogel layer upon saturation of an exudate from the wound, and applying a replacement second aerogel layer to the first aerogel layer, such that the replacement second aerogel layer removes exudate from the first aerogel layer.

Another embodiment of the present invention provides a method of treating a wound as in any embodiment above, wherein the aerogel wound dressing includes a sensing component.

Another embodiment of the present invention provides a method of treating a wound as in any embodiment above, further including the aerogel wound dressing includes a sensing component.

Another embodiment of the present invention provides a method of treating a wound as in any embodiment above, further including removing the aerogel wound dressing from the wound when the sensing component indicates the presence of a pathogen.

Another embodiment of the present invention provides a method of treating a wound as in any embodiment above, further including applying a treatment to the aerogel wound dressing when the sensing component indicates the presence of a pathogen.

Another embodiment of the present invention provides a method of treating a wound as in any embodiment above, wherein the aerogel wound dressing is a multi-layered wound dressing including a first aerogel layer and a second aerogel layer in contact with the first aerogel layer, wherein the second aerogel layer is removable from the aerogel wound dressing.

Another embodiment of the present invention provides a method of treating a wound as in any embodiment above, further including removing the second aerogel layer upon saturation of an exudate from the wound.

Another embodiment of the present invention provides a method of treating a wound as in any embodiment above, wherein further including applying a replacement second aerogel layer to the first aerogel layer, such that the replacement second aerogel layer is in contact with the first aerogel layer and removes exudate from the first aerogel layer.

Another embodiment of the present invention provides a method of treating a wound as in any embodiment above, wherein the aerogel wound dressing is applied to the wound for 20 days or greater.

### BRIEF DESCRIPTION OF THE DRAWINGS

FIG. 1 is a representative side view of a cross section of an embodiment of an aerogel wound dressing applied to a wound;

FIG. 2A shows an aerogel wound dressing according to an embodiment of the present invention including a sensing component;

FIG. 2B shows an aerogel wound dressing according to an embodiment of the present invention including a sensing component being subjected to artificial wound fluid;

FIG. 2C shows an aerogel wound dressing according to an embodiment of the present invention including a sensing component that has undergone a color change after being subjected to an increase in pH by the artificial wound fluid.

### DETAILED DESCRIPTION OF ILLUSTRATIVE EMBODIMENTS

The present invention relates to the use of aerogel in wound dressings as means of improving the function of wound dressings to promote wound healing and improve wound care. The aerogel wound dressings provide highly absorbent dressings that require less frequent replacement and remove the need for cumbersome Negative Pressure Wound Therapy (NPWT) devices in order to enhance the wound healing environment. In some embodiments, the present invention relates to methods of treating wounds using wound dressings including aerogel and aerogel composites.

Aerogels are a class of materials that have low density and high porosity, typically consisting of 90-99% air by volume. Aerogels are known for unique physical properties, such as low thermal conductivity, high surface area, and high specific surface area. These properties are attributed to porous microstructures created during the formation of aerogels. The porous microstructure of aerogels is a defining characteristic. The pore structure of aerogels can be controlled by adjusting the synthesis parameters, such as the precursor concentration, solvent choice, and drying conditions. This allows for the creation of aerogels with a wide range of pore sizes, from microporous to mesoporous to macroporous.

As used herein, pore size may refer to an average pore size. As one of ordinary skill in the art understands, the porous structure of an aerogel will include a distribution of pore sizes. As used herein, pore size may refer to when a majority of the pores of a particular aerogel composition fall within a stated range.

Microporous aerogels have pore sizes less than 2 nm and are often used for applications such as gas separation and catalysis. Mesoporous aerogels have pore sizes between 2 and 50 nm and are commonly used in applications such as energy storage and drug delivery. Macroporous aerogels have pore sizes greater than 50 nm and are often used for applications such as thermal insulation and sound absorption

In one or more embodiments, aerogels suitable for use in wound dressings according to the present invention include mesopores. In one or more embodiments, the mesopores have a pore hole size of from about 2 nm to about 50 nm.

In one or more embodiments, aerogels suitable for use in wound dressings according to the present invention include macropores. In one or more embodiments, the macropores have a pore hole size of from about 50 nm to about 500 nm. In other embodiments, about 50 nm to about 450 nm. In other embodiments, about 50 nm to about 400 nm. In other embodiments, about 50 nm to about 350 nm. In other embodiments, about 50 nm to about 300 nm. In other embodiments, about 50 nm to about 250 nm. In other embodiments, about 50 nm to about 200 nm.

Pore size is a critical parameter in determining the capillary action of an aerogel. Capillary action is the ability of a liquid to flow through narrow spaces, such as the interconnected pores of an aerogel. The driving force for capillary action is the surface tension of the liquid, which pulls the liquid into the narrow spaces. In general, smaller pores result in greater capillary action. This is because smaller pores have a higher surface area-to-volume ratio, which increases the surface tension at the liquid-gel interface. As a result, liquids can flow more easily through smaller pores, even against gravity.

In the context of wound healing, the capillary action of an aerogel is important for promoting a moist wound healing environment while removing wound exudate from the wound surface. The interconnected pores of the aerogel can absorb wound exudate and hold it within the aerogel. The capillary action of the aerogel can help to keep the wound moist, which is beneficial for wound healing.

It is also important to control the pore size to prevent excessive fluid retention, which can lead to maceration of the surrounding healthy tissue. Therefore, the pore size of an aerogel for use in a wound dressing should be optimized to achieve the desired level of capillary action, while also preventing fluid overload.

Advantageously, aerogels suitable for use in embodiments of the present invention serve as an air permeable barrier against bacteria and viruses. The interconnected pores of aerogels used according to some embodiments of the present invention allow for oxygen to diffuse through the aerogel and reach the wound. The same interconnected pores filter, or reject, bacterial or viral infiltration of the aerogel. This filtration protects the wound from coming into contact with bacteria or viruses present in the environment, thereby protecting the wound from infections caused by the bacteria or viruses.

In one or more embodiments, the aerogel has a filtration efficiency of nanoparticles, sized 25 nm to 250 nm of about 98.9% or greater. In other embodiments, about 99.0% or greater. In other embodiments, about 99.1% or greater. In other embodiments, about 99.2% or greater. In other embodiments, about 99.3% or greater. In other embodiments, about 99.4% or greater. In other embodiments, about 99.5% or greater. In other embodiments, about 99.6% or greater. In other embodiments, about 99.7% or greater. In other embodiments, about 99.8% or greater. In other embodiments, about 99.9% or greater.

In one or more embodiments, the aerogel has a filtration efficiency of E. Coli bacteria of about 95% or greater. In other embodiments, about 96% or greater. In other embodiments, about 97% or greater. In other embodiments, about 98% or greater. In other embodiments, about 99% or greater.

Aerogels are formed by turning a gel into an aerogel. The material composition of aerogel suitable for use in the present invention is determined by the gels and processes used for turning the gel into an aerogel. As discussed above, the gels and processes used also affect the resulting porous microstructures of the obtained aerogels.

The selection of materials is also dependent upon the use of the aerogel in a wound dressing as a biomedical application. In accordance with the objectives of the present invention, suitable aerogel compositions are safe for use on wounds, including chronic wounds. Further, the selection of materials for aerogel formation is dependent on the mechanical properties required for use in a wound dressing. In some embodiments, aerogel compositions may include biopolymers. In other embodiments, aerogel compositions may include a mixture of biopolymers. In other embodiments, aerogel compositions may include a biopolymer gel grown inside another.

As used herein, biopolymer may refer to polymers that are compatible for animal use. Biopolymer may also refer to polymers that are derived from biomass or other natural sources including natural polymers produced by the cells of living organisms. The biocompatibility of the aerogel material allows the dressing to be used on a wide range of wounds, including chronic wounds that require long-term treatment. The aerogel layer provides a highly absorbent and breathable layer that conforms to the contours of the wound surface, promoting a moist wound healing environment that is conducive to tissue regeneration. The use of a biocompatible aerogel material further enhances the effectiveness of the dressing by minimizing the risk of infection or other complications that can arise from the use of non-biocompatible materials.

In some embodiments, materials suitable for aerogel compositions include biopolymers. In some embodiments, biopolymers include polynucleotides, polypeptides, and polysaccharides. In some embodiments, polypeptides includes proteins and amino acids, including collagen, actin, and fibrin. In some embodiments, polysaccharides include starch, cellulose, and alginate. Other biopolymers include natural rubbers, suberin, lignin, cutin, cutan, and melanin. In some embodiments, materials suitable for aerogel compositions include chitosan, cellulose, cellulose nanocrystals, lignin, and combinations of two or more.

In some embodiments, materials suitable for aerogel compositions include biocompatible polymers, including those produced synthetically.

In some embodiments, the mechanical properties of the aerogel may be improved by forming composite aerogels. In these and other embodiments, a sol network, or framework, may first be formed. A sol of a second gel may then be diffused within the framework, which is then gelled. Advantageously, this composite structure reduces shrinkage of the aerogel during the solvent exchange and leads to improved nucleation and growth. In such embodiments, the framework may comprise synthetic fibers. Synthetic fibers suitable for use in the present invention include, without limitation, Kevlar.

According to some embodiments, the present invention is an aerogel comprising a layer of aerogel material that includes a sensing component. The sensing component is designed to provide real-time feedback on the wound environment, allowing for monitoring of the healing process and the ability to make informed decisions about treatment.

In one embodiment, the sensing component may be a pH sensor that detects changes in the acidity or alkalinity of the wound environment. In another embodiment, the sensing component may be a temperature sensor that measures the temperature of the wound surface. Other possible sensing components may include pressure sensors, oxygen sensors, or sensors that detect the presence of specific bacteria, viruses, or other pathogens.

In some embodiments, the sensing component in these embodiments can be an additive that changes color in response to pH changes. According to the objectives of the present invention, additives to the aerogel composition should be biocompatible. This type of additive is commonly known as a pH indicator. A pH indicator can be incorporated into the aerogel either during the aerogel synthesis process or as a post-processing step. The sensing additive may be included during gel formation or during solvent exchange and drying of the gel. The sensing additive may also be included in the aerogel through emulsion templating, precipitation, or precipitation using a non-solvent during solvent exchange steps. The pH indicator can be chosen based on its sensitivity to a specific pH range, which can be tailored to the needs of the wound healing application.

When the aerogel wound dressing is in contact with the wound bed, the pH indicator can detect changes in the pH level of the wound exudate. The pH indicator will change color in response to the pH change, which can be visually detected. This can provide a simple and non-invasive way to monitor the pH level of the wound over time.

In some embodiments, the color change of the pH indicator can be correlated with specific pH values, which can be determined through calibration experiments. This can provide a quantitative measurement of the pH level, which can be recorded and tracked over time to monitor the progress of wound healing.

In summary, in some embodiments of the aerogel wound dressing of claim 1, the sensing component is designed to measure pH levels in the wound bed using a pH indicator additive. The pH indicator changes color in response to pH changes, providing a simple and non-invasive way to monitor the pH level of the wound over time.

In some embodiments of the sensing agent is a compound that exhibits keto-enol tautomerism. Tautomers are isomers of a molecule that differ in the placement of a proton and the double bond. Keto-enol tautomerism is a type of tautomerism that involves the transfer of a proton between the carbonyl group (keto) and the hydroxyl group (enol) of an organic molecule.

As discussed above, the sensing agent may be incorporated into the aerogel layer of the wound dressing as an additive. When the wound dressing is in contact with wound exudate, the pH indicator will undergo a proton transfer between the keto and enol forms in response to changes in the pH level. This proton transfer causes a change in the absorption spectrum of the pH indicator, resulting in a color change that can be visually detected.

The pH indicator is not particularly limited and one of ordinary skill in the art will be able to select a suitable pH indicator without undue experimentation. In various embodiments, a suitable pH indicator may include, without limitation, curcumin. Advantageously, curcumin is known to be beneficially affect oxidative and inflammatory conditions.

As shown in FIGS. 2A through 2C an aerogel wound dressing according to an embodiment of the present invention demonstrates the change of color of the wound dressing when subjected to an artificial wound fluid that increases the pH of the wound dressing. In FIG. 2A, an unused aerogel wound dressing is shown possessing a yellow color. In FIG. 2B, the aerogel wound dressing is subjected artificial infected wound fluid which increases the pH of the aerogel wound dressing, and is beginning to undergo a color change of yellow to red. In FIG. 2C, the entirety of the aerogel wound dressing is a red-brown color indicating the change caused by the absorption of the artificial wound exudate.

Other additives may be included in aerogel compositions suitable for use in the present invention. For example, aerogel compositions may be used a means of delivering pharmaceutical therapies to a wound. The pharmaceutical therapies are not particularly limited and one of ordinary skill in the art will be able to select a suitable pharmaceutical therapy without undue experimentation. In various embodiments, a suitable pharmaceutical therapy may include, without limitation, biocides and antibiotics.

In some embodiments, the two aerogels may be used in wound dressings according to the present invention. In these and other embodiments, the two aerogels may be arranged as a base layer and a top layer, also referred to as a first layer and a second layer, respectively. In these and other embodiments, the base layer functions to remove exudate from the wound surface, while the top layer removes exudate from the base layer. Without wishing to be bound by theory, it is believed that capillary action resulting from the pore size of the aerogels provides the driving force necessary to remove exudate from the base layer, through the interface between the base layer and the top layer, and into the top layer. In these and other embodiments, the capillary action may be provided by the base layer having a porous structure with a larger pore size than the pore size of the porous structure of the top layer.

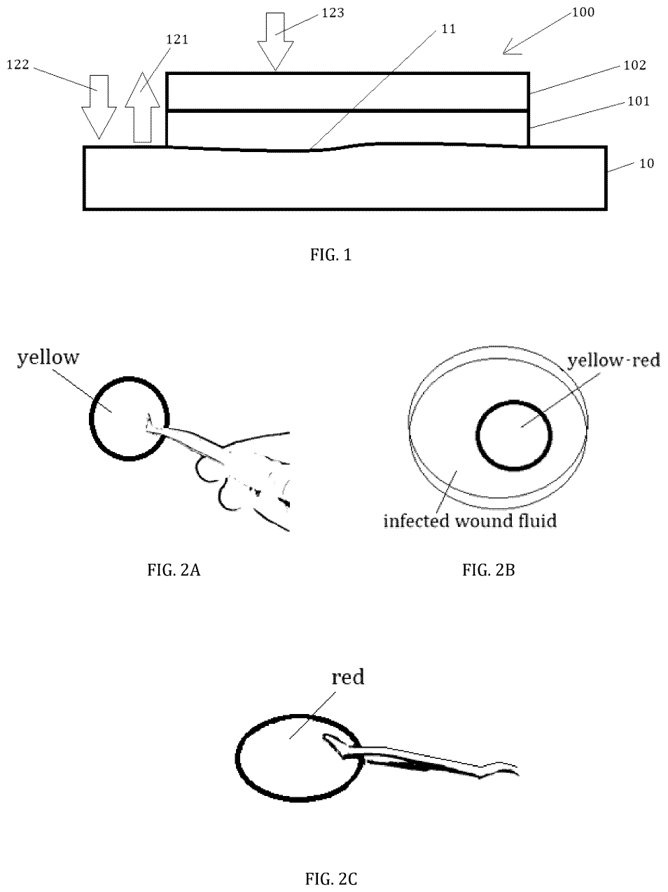

Referring now to FIG. 1, a multi-layered aerogel wound dressing is shown, generally indicated by the numeral 100. Multi-layered aerogel wound dressing 100 includes first layer 101, also referred to as base layer, and second layer 102, also referred to as top layer. Multi-layered aerogel wound dressing 100 is applied to wound surface 11 of subject 10, such that first layer 102 is in contact with wound surface 11. First layer 101 includes a macroporous structure. Second layer 102 includes a mesoporous structure. Arrow 121 is representative of the flow of exudate from wound surface 11 through first layer 101 and into second layer 102. Arrow 122 is representative of the air permeability multi-layered aerogel wound dressing 100, wherein oxygen may permeate first layer 101 and second layer 102 to reach wound surface 11. Arrow 123 is representative of the rejection of pathogens, including bacteria and viruses, by multi-layered aerogel wound dressing 100 as result of the mesoporous structure of second layer 102.

In embodiments according to the present invention, the base layer comprises an aerogel having macropores. In these and other embodiments, the top layer comprises an aerogel having mesopores. In such embodiments, the difference in the pore size between the two layers allows for exudate to collect in the top layer, while maintain moisture in the bottom layer. Advantageously, the removal of exudate prevents the formation of biofilms, while the two layers of aerogel protect the wound from bacteria, viruses, and other pathogens. Further, the two-layer structure provides for the removal and replacement of the top layer upon saturation of the top layer with exudate. In these and other embodiments, the base layer may remain in contact with the wound while the top layer is removed and replaced with a clean top layer to provide continuous removal of exudate without irritation to the wound caused by replacement of wound dressing.

In embodiments according to the present invention, one or more of the base layer and top layer may include a sensing agent according to the description above.

The role of a wound dressing is to provide the optimum conditions for wound healing, whilst protecting the wound from further trauma and invasion by pathogenic microorganisms. It is also important that the dressings can be removed atraumatically, so as to prevent further damage to the wound surface during dressing changes. In embodiments according to the present invention provides for aerogel wound dressings including at least an aerogel layer. In some embodiments, the aerogel layer includes an aerogel composition according to any of the descriptions included above.

In some embodiments the aerogel layer of the aerogel wound dressing is a bottom layer in contact with the wound.

In some embodiments, aerogel wound dressings may further include a fiber dressing as a bottom layer in contact with the wound. In other embodiments aerogel wound dressings may include a foam dressing as a bottom layer in contact with the wound.

In some embodiments, aerogel wound dressings includes two layers formed from aerogel compositions according to any of the descriptions included above. In these and other embodiments, the aerogel wound dressing may include a first aerogel layer as a base layer and a second aerogel layer as a top layer. In these and other embodiments, the top layer may be removable from the base layer. In these and other embodiments, the top layer may have a pore size that is lesser than the pore size of the base layer. In these and other embodiments, the base layer may include a sensing component for providing insight to the wound environment. In these and other embodiments, the sensing component may include any sensing component as described above.

In some embodiments, aerogel wound dressings may be held in place at a wound using medical adhesives.

In some embodiments, aerogel wound dressings according to the present invention may be considered as a primary wound dressing. In these and other embodiments, secondary wound dressings known in the art may be used to secure the aerogel wound dressing in place. In these and other embodiments, the secondary dressing may be removed to access the primary wound dressing, which may include monitoring the sensing component or removing and replacing the second layer of a multilayer aerogel wound dressing.

In some embodiments, aerogel wound dressings according to the present invention are sized according to the type of wound intended to be treated. In some embodiments, aerogel wound dressings according to the present invention are sized according to known standards in the art of wound dressings. A properly sized wound dressing ensures coverage of the entire surface area of the wound to be treated by the wound dressing.

In some embodiments, aerogel wound dressings according to the present invention absorb 10 times or greater weight of fluid per unit weight of aerogel wound dressing. In other embodiments, 15 times or greater weight of fluid per unit weight of aerogel wound dressing. In other embodiments, 20 times or greater weight of fluid per unit weight of aerogel wound dressing. In other embodiments, 25 times or greater weight of fluid per unit weight of aerogel wound dressing. In other embodiments, 30 times or greater weight of fluid per unit weight of aerogel wound dressing. In other embodiments, 35 times or greater weight of fluid per unit weight of aerogel wound dressing. In other embodiments, 40 times or greater weight of fluid per unit weight of aerogel wound dressing. In other embodiments, 45 times or greater weight of fluid per unit weight of aerogel wound dressing. In other embodiments, 50 times or greater weight of fluid per unit weight of aerogel wound dressing. In other embodiments, 55 times or greater weight of fluid per unit weight of aerogel wound dressing. In other embodiments, 60 times or greater weight of fluid per unit weight of aerogel wound dressing.

In some embodiments including a removable second layer in contact with a first layer, the removable second layer may be the same thickness as the first layer. In other embodiments, the removable second layer may be of a greater thickness than the first layer. It will be readily understood by those of ordinary skill in the art, that thickness of the layer directly affects the amount of wound exudate that is absorbed by the layer.

In use, aerogel wound dressings according to the present invention provide a highly absorbent and breathable dressing that conforms to the contours of the wound surface. The sensing component, when present, provides real-time feedback on the wound environment, allowing for the monitoring of the healing process. The unique combination of aerogel material and sensing component provides a versatile and effective wound dressing that can be used to treat a wide range of wounds, including chronic wounds that are difficult to heal using traditional treatments.

Embodiments according to the present invention provide methods for treating a wound using an aerogel wound dressing. In some embodiments, methods of treating a wound may include applying an aerogel wound dressing to a wound. In these and other embodiments, applying may include applying an optional secondary wound dressing to secure the aerogel wound dressing in place. In these and other embodiments, the aerogel wound dressing may remove exudate from the wound as described above. In some embodiments, the aerogel wound dressing may include a sensing component. In these and other embodiments the method may include monitoring the sensing component for the presence of a pathogen. As discussed above, the presence of determining whether a pathogen is present in the wound environment may be indicated by the sensing component. In these and other embodiments, the method may further include removing the aerogel wound dressing from the wound when the sensing component indicates the presence of a pathogen. Removal allows for the wound to be treated as required.

In embodiments where the method includes applying an aerogel wound dressing including at least a base aerogel layer and top aerogel layer, the method may further include removing the top aerogel layer upon saturation of the top aerogel layer of exudate from the wound. In these and other embodiments, the method may further include replacement of the top aerogel layer with a replacement top aerogel layer such that exudate may be continued to be removed from the wound. In these and other embodiments, the method may include leaving the base layer of the aerogel wound dressing in contact with the wound for 7 days or greater. In other embodiments, 14 days or greater. In other embodiments, 15 days or greater. In other embodiments, 16 days or greater. In other embodiments, 17 days or greater. In other embodiments, 18 days or greater. In other embodiments, 19 days or greater. In other embodiments, 20 days or greater. In other embodiments, 21 days or greater.

Embodiments of the present invention, as described above, may be manufactured according to the following methods. In some embodiments, gel compositions are formed according to the desired final characteristics of the resulting aerogel. In these and other embodiments, this may include providing any desired additives during gel formation. In these and other embodiments, gel formation includes providing a sol which undergoes sol-gel transition. In some embodiments, a second sol of a second gel may be diffused within a sol network or framework produced from a first gel. In such embodiments, the second sol is gelled after diffusion. The shape of the resulting aerogel may be determined by the mold in which the gel compositions set. Various molds may be used to produce different aerogels according to the type of wound the resulting aerogel wound dressing is intended to treat.

After the gel has set within a mold, gels may be subjected to solvent exchange. In some embodiments, additives may be included in the gel during the step of solvent exchange. In some embodiments, following the step of solvent exchange, the gel may undergo a step of supercritical drying.

Other techniques known in the art may be suitable for creating aerogel compositions according to the embodiments described above.

In light of the foregoing, it should be appreciated that the present invention significantly advances the art by providing wound dressings and methods of treating wounds that are structurally and functionally improved in a number of ways. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

### EXAMPLES

To illustrate and reduce the invention to practice, aerogel wound dressings of the present invention were synthesized as set forth above and were evaluated to more fully determine the following characteristics.

Specimens of various wound dressings were tested to determine their absorption capacities. The wound dressings include Duoderm, Algisite, Drawtex, Aquacel, and an aerogel wound dressing according an embodiment of the present invention. Each specimen was immersed in 100 ml of water, and the mass gain was recorded over time. The results of this are reported in Table I.

TABLE I
Absorption Capacity Results

| | | Specimen | Specimen | Absorption |
|---|---|---|---|---|
| | Specimen | mass after | mass after | per unit |
| Specimen | mass (g) | 5 hours (g) | 18 hours (g) | weight |
| Duoderm | 0.1750 | 2.9233 | 2.9479 | 17 |
| Algisite | 0.5183 | 1.1653 | 1.7669 | 3 |
| Drawtex | 0.4218 | 2.8208 | 2.8427 | 7 |
| Aquacel | 1.0618 | 11.9146 | 15.9706 | 15 |
| Aerogel | 0.0264 | 1.2732 | 1.3499 | 51 |
| Wound | | | | |
| Dressing | | | | |

The aerogel wound dressing according to the present invention demonstrates superior absorption per unit weight relative to other types of wound dressings.

Specimens of the wound dressings were tested to determine their filtration efficiency of nanometer sized impurities. This test simulates the performance of the dressings in the presence of microorganisms. Specimens of Algisite, Drawtex, Aquacel, and an aerogel wound dressing according to an embodiment of the present invention were subjected to a solution including NaCl nanoparticles, having a size of 25 nm to 250 nm, at a flow of 20 liters/min. The results of this test are reported in Table II.

TABLE II
Filtration Efficiency Results

| | Filtration |
|---|---|
| Specimen | Efficiency (%) |
| Algisite | 9.43 ± 0.8 |
| Drawtex | 23.03 ± 0.4 |
| Aqualcel | 22.73 ± 0.7 |
| Aerogel Wound | 99.46 ± 0.5 |
| Dressing | |

The foregoing experimental parameters and set-ups were used to generate the data and information provided herein in support of the claimed invention. Although the invention has been described in detail with particular reference to certain embodiments detailed herein, other embodiments can achieve the same results. Variations and modifications of the present invention will be obvious to those skilled in the art and the present invention is intended to cover in the appended claims all such modifications and equivalents

What is claimed is:

1. An aerogel wound dressing comprising:
a first aerogel layer, wherein the first aerogel layer includes a sensing component.

2. The aerogel wound dressing of claim 1, wherein the sensing component indicates a change in pH of a wound exudate absorbed by the aerogel layer.

3. The aerogel wound dressing of claim 2, wherein the sensing component changes color because of the change in pH of the wound exudate absorbed by the aerogel layer.

4. The aerogel wound dressing of claim 1, wherein the sensing component comprises curcumin.

5. The aerogel wound dressing of claim 1, wherein the sensing component is added to the first aerogel layer during formation of the first aerogel layer.

6. The aerogel wound dressing of claim 1, wherein the sensing component comprises a probe connected to an external sensing device.

7. The aerogel wound dressing of claim 1, wherein the first aerogel layer absorbs 10 times or greater exudate by weight per weight of first aerogel layer.

8. The aerogel wound dressing of claim 1, further comprising:
a second aerogel layer in contact with the first aerogel layer, wherein the second aerogel layer is removable from the aerogel wound dressing.

9. The aerogel wound dressing of claim 8, wherein the second aerogel layer absorbs fluids absorbed by the first aerogel layer.

10. The aerogel wound dressing of claim 8, wherein the first aerogel layer includes a first plurality of interconnected pores, and wherein the second aerogel layer includes a second plurality of interconnected pores.

11. The aerogel wound dressing of claim 10, wherein an average pore size of the first plurality of interconnected pores is larger than an average pore size of the second plurality of interconnected pores.

12. The aerogel wound dressing of claim 8, wherein the first aerogel layer includes a sensing component.

13. The aerogel wound dressing of claim 8, wherein the aerogel wound dressing is arranged such that the second aerogel layer is user replaceable upon saturation and removal of the second aerogel layer.

14. The aerogel wound dressing of claim 13, wherein the aerogel wound dressing is arranged such that the first aerogel layer is arranged to remain in place upon removal of the second aerogel layer.

15. The aerogel wound dressing of claim 8, wherein the first aerogel layer includes a sensing component.

16. The aerogel wound dressing of claim 8, wherein pathogenic agents cannot pass through the first aerogel layer.

17. The aerogel wound dressing of claim 8, wherein the first aerogel layer is formed from at least one of cellulose, cellulose nanocrystals, chitosan, Kevlar, lignin, or any combination thereof, and wherein the second aerogel layer is formed from at least one of cellulose, cellulose nanocrystals, chitosan, Kevlar, lignin, or any combination thereof

18. A method of treating a wound, the method comprising:
applying an aerogel wound dressing to a wound.

19. The method of claim 18, wherein the aerogel wound dressing includes a sensing component, the method further comprising:
monitoring the sensing component for the presence of a pathogen;
removing the aerogel wound dressing from the wound when the sensing component indicates the presence of a pathogen; and
applying a treatment to the aerogel wound dressing when the sensing component indicates the presence of a pathogen.

20. The method of claim 19, wherein the aerogel wound dressing is a multilayered aerogel wound dressing comprising:
a first aerogel layer, including the sensing component; and
a second aerogel layer in contact with the first aerogel layer, wherein the second aerogel layer is removable from the aerogel wound dressing; and
the method further comprising:
removing the second aerogel layer upon saturation of an exudate from the wound; and
applying a replacement second aerogel layer to the first aerogel layer, such that the replacement second aerogel layer removes exudate from the first aerogel layer.
